SEQUENCE LISTING (17 sequences)

SEQ ID NO: 1 - DNA, 11 bp, Artificial Sequence
  Description of Artificial Sequence: Synthetic oligonucleotide
  modified_base (3)..(3): 7-deaza-dG
  modified_base (7)..(7): 7-deaza-dG
  modified_base (11)..(11): 7-deaza-dG
tcgtacgtacg

SEQ ID NO: 2 - DNA, 11 bp, Artificial Sequence
  Description of Artificial Sequence: Synthetic oligonucleotide
  modified_base (3)..(3): 7-deaza-dG
  misc_feature (6)..(7): phosphodiester linkage
  modified_base (7)..(7): 7-deaza-dG
  modified_base (11)..(11): 7-deaza-dG
tcgtacgtacg

SEQ ID NO: 3 - DNA, 11 bp, Artificial Sequence
  Description of Artificial Sequence: Synthetic oligonucleotide
  modified_base (3)..(3): 7-deaza-dG
  misc_feature (6)..(7): phosphodiester linkage
  modified_base (7)..(7): 7-deaza-dG
  misc_feature (10)..(11): phosphodiester linkage
  modified_base (11)..(11): 2′-O-methyl-G
tcgtacgtacg

SEQ ID NO: 4 - DNA, 11 bp, Artificial Sequence
  Description of Artificial Sequence: Synthetic oligonucleotide
  modified_base (3)..(3): arabino-G
  misc_feature (6)..(7): phosphodiester linkage
  modified_base (7)..(7): arabino-G
  misc_feature (10)..(11): phosphodiester linkage
  modified_base (11)..(11): arabino-G
tcgtacgtacg

SEQ ID NO: 5 - DNA, 11 bp, Artificial Sequence
  Description of Artificial Sequence: Synthetic oligonucleotide
  modified_base (3)..(3): 7-deaza-dG
  misc_feature (5)..(6): phosphodiester linkage
  modified_base (6)..(6): 7-deaza-dG
  misc_feature (8)..(9): phosphodiester linkage
  modified_base (9)..(9): 2′-O-methyl-G
tcgtcgacgat

SEQ ID NO: 6 - DNA, 11 bp, Artificial Sequence
  Description of Artificial Sequence: Synthetic oligonucleotide
  modified_base (3)..(3): arabino-G
  misc_feature (5)..(6): phosphodiester linkage
  misc_feature (6)..(7): phosphodiester linkage
  modified_base (7)..(7): arabino-G
  misc_feature (7)..(8): phosphodiester linkage
  modified_base (11)..(11): arabino-G
tcgtacgtacg

SEQ ID NO: 7-9 - skipped (3 entries)

SEQ ID NO: 10 - DNA, 11 bp, Artificial Sequence
  Description of Artificial Sequence: Synthetic oligonucleotide
  misc_feature (2)..(3): phosphodiester linkage
  modified_base (3)..(3): 7-deaza-dG
  misc_feature (3)..(4): phosphodiester linkage
  misc_feature (6)..(7): phosphodiester linkage
  modified_base (7)..(7): 7-deaza-dG
  misc_feature (10)..(11): phosphodiester linkage
  modified_base (11)..(11): 7-deaza-dG
tcgaacgttcg

SEQ ID NO: 11 - DNA, 11 bp, Artificial Sequence
  Description of Artificial Sequence: Synthetic oligonucleotide
  misc_feature (1)..(2): phosphodiester linkage
  modified_base (3)..(3): 7-deaza-dG
  misc_feature (3)..(4): phosphodiester linkage
  misc_feature (6)..(7): phosphodiester linkage
  modified_base (7)..(7): 7-deaza-dG
  misc_feature (10)..(11): phosphodiester linkage
  modified_base (11)..(11): 7-deaza-dG
tcgaacgttcg

SEQ ID NO: 12 - DNA, 11 bp, Artificial Sequence
  Description of Artificial Sequence: Synthetic oligonucleotide
  misc_feature (2)..(3): phosphodiester linkage
  modified_base (3)..(3): 7-deaza-dG
  misc_feature (3)..(4): phosphodiester linkage
  misc_feature (6)..(7): phosphodiester linkage
  modified_base (7)..(7): 7-deaza-dG
  misc_feature (10)..(11): phosphodiester linkage
  modified_base (11)..(11): 7-deaza-dG
tcgaacgttcg

SEQ ID NO: 13 - DNA, 11 bp, Artificial Sequence
  Description of Artificial Sequence: Synthetic oligonucleotide
  misc_feature (2)..(3): phosphodiester linkage
  modified_base (3)..(3): arabino-G
  misc_feature (3)..(4): phosphodiester linkage
  misc_feature (6)..(7): phosphodiester linkage
  modified_base (7)..(7): arabino-G
  misc_feature (10)..(11): phosphodiester linkage
  modified_base (11)..(11): arabino-G
tcgaacgttcg

SEQ ID NO: 14 - DNA, 11 bp, Artificial Sequence
  Description of Artificial Sequence: Synthetic oligonucleotide
  misc_feature (2)..(3): phosphodiester linkage
  modified_base (3)..(3): 7-deaza-dG
  misc_feature (3)..(4): phosphodiester linkage
  misc_feature (5)..(6): phosphodiester linkage
  modified_base (6)..(6): 7-deaza-dG
  misc_feature (8)..(9): phosphodiester linkage
  modified_base (9)..(9): 2′-O-methyl-G
tcgtcgacgat

SEQ ID NO: 15 - DNA, 11 bp, Artificial Sequence
  Description of Artificial Sequence: Synthetic oligonucleotide
  misc_feature (2)..(3): phosphodiester linkage
  modified_base (3)..(3): 7-deaza-dG
  misc_feature (3)..(4): phosphodiester linkage
  misc_feature (6)..(7): phosphodiester linkage
  modified_base (7)..(7): 7-deaza-dG
  misc_feature (10)..(11): phosphodiester linkage
  modified_base (11)..(11): 2′-O-methyl-G
tcgaacgttcg

SEQ ID NO: 16 - DNA, 11 bp, Artificial Sequence
  Description of Artificial Sequence: Synthetic oligonucleotide
  misc_feature (1)..(2): phosphodiester linkage
  modified_base (3)..(3): 7-deaza-dG
  misc_feature (3)..(4): phosphodiester linkage
  misc_feature (6)..(7): phosphodiester linkage
  modified_base (7)..(7): 7-deaza-dG
  misc_feature (10)..(11): phosphodiester linkage
  modified_base (11)..(11): 2′-O-methyl-G
tcgaacgttcg

SEQ ID NO: 17 - DNA, 11 bp, Artificial Sequence
  Description of Artificial Sequence: Synthetic oligonucleotide
  misc_feature (2)..(3): phosphodiester linkage
  modified_base (3)..(3): arabino-G
  misc_feature (3)..(4): phosphodiester linkage
  misc_feature (6)..(7): phosphodiester linkage
  modified_base (7)..(7): arabino-G
  misc_feature (10)..(11): phosphodiester linkage
  modified_base (11)..(11): arabino-G
tcgtacgtacg

(19) United States
(12) Patent Application Publication
(10) Pub. No.: US 2011/0293565 A1
(43) Pub. Date: Dec. 1, 2011

(54) NOVEL SYNTHETIC AGONISTS OF TLR9

(75) Inventors: Ekambar Kandimalla, Southboro, MA (US); Mallikarjuna Putta, Burlington, MA (US); Daqing Wang, Bedford, MA (US); Dong Yu, Westboro, MA (US); Lakshmi Bhagat, Framingham, MA (US); Sudhir Agrawal, Shrewsbury, MA (US)

(73) Assignee: IDERA PHARMACEUTICALS, INC., Cambridge, MA (US)

(21) Appl. No.: 12/695,883

(22) Filed: Jan. 28, 2010

Related U.S. Application Data
(60) Provisional application No. 61/148,527, filed on Jan. 30, 2009.
(60) Provisional application No. 61/280,065, filed on Oct. 29, 2009.

(51) Int. Cl. A61K 39/00; A61P 35/00; A61P 29/00; A61K 38/21; A61P 17/00; A61P 37/08; A61P 11/06; C07H 21/04; A61P 31/00

(52) U.S. Cl. 424/85.7; 536/23.1; 424/184.1

(57) ABSTRACT

The invention relates to synthetic chemical compositions that are useful for modulation of Toll-Like Receptor (TLR)-mediated immune responses. In particular, the invention relates to agonists of Toll-Like Receptor 9 (TLR9) that generate unique cytokine and chemokine profiles.

This application claims the benefit of priority from U.S. Provisional Patent Applications No. 61/148,527, filed on Jan. 30, 2009 and 61/280,065 filed on Oct. 29, 2009, the disclosure of which is explicitly incorporated by reference herein.

### BACKGROUND OF THE INVENTION

1. Field of the Invention

The invention relates to synthetic chemical compositions that are useful for modulation of Toll-Like Receptor (TLR)-mediated immune responses. In particular, the invention relates to agonists of Toll-Like Receptor 9 (TLR9) that generate unique cytokine and chemokine profiles.

2. Summary of the Related Art

Toll-like receptors (TLRs) are present on many cells of the immune system and have been shown to be involved in the innate immune response (Hornung, V. et al., (2002) J. Immunol. 168:4531-4537). In vertebrates, this family consists of eleven proteins called TLR1 to TLR11 that are known to recognize pathogen associated molecular patterns from bacteria, fungi, parasites, and viruses (Poltorak, a. et al. (1998) Science 282:2085-2088; Underhill, D. M., et al. (1999) Nature 401:811-815; Hayashi, F. et. al (2001) Nature 410:1099-1103; Zhang, D. et al. (2004) Science 303:1522-1526; Meier, A. et al. (2003) Cell. Microbiol. 5:561-570; Campos, M. A. et al. (2001) J. Immunol. 167: 416-423; Hoebe, K. et al. (2003) Nature 424: 743-748; Lund, J. (2003) J. Exp. Med. 198:513-520; Heil, F. et al. (2004) Science 303:1526-1529; Diebold, S. S., et al. (2004) Science 303:1529-1531; Hornung, V. et al. (2004) J. Immunol. 173:5935-5943).

TLRs are a key means by which vertebrates recognize and mount an immune response to foreign molecules and also provide a means by which the innate and adaptive immune responses are linked (Akira, S. et al. (2001) Nature Immunol. 2:675-680; Medzhitov, R. (2001) Nature Rev. Immunol. 1:135-145). Some TLRs are located on the cell surface to detect and initiate a response to extracellular pathogens and other TLRs are located inside the cell to detect and initiate a response to intracellular pathogens.

TLR9 is known to recognize unmethylated CpG motifs in bacterial DNA and in synthetic oligonucleotides. (Hemmi, H. et al. (2000) Nature 408:740-745). Other modifications of CpG-containing phosphorothioate oligonucleotides can also affect their ability to act as modulators of immune response through TLR9 (see, e.g., Zhao et al., Biochem. Pharmacol. (1996) 51:173-182; Zhao et al. (1996) Biochem Pharmacol. 52:1537-1544; Zhao et al. (1997) Antisense Nucleic Acid Drug Dev. 7:495-502; Zhao et al (1999) Bioorg. Med. Chem. Lett. 9:3453-3458; Zhao et al. (2000) Bioorg. Med. Chem. Lett. 10:1051-1054; Yu, D. et al. (2000) Bioorg. Med. Chem. Lett. 10:2585-2588; Yu, D. et al. (2001) Bioorg. Med. Chem. Lett. 11:2263-2267; and Kandimalla, E. et al. (2001) Bioorg. Med. Chem. 9:807-813). Naturally occurring agonists of TLR9 have been shown to produce anti-tumor activity (e.g. tumor growth and angiogenesis) resulting in an effective anti-cancer response (e.g. anti-leukemia) (Smith, J. B. and Wickstrom, E. (1998) J. Natl. Cancer Inst. 90:1146-1154). In addition, TLR9 agonists have been shown to work synergistically with other known anti-tumor compounds (e.g. cetuximab, irinotecan) (Vincenzo, D., et al. (2006) Clin. Cancer Res. 12(2):577-583).

Certain TLR9 agonists are comprised of 3′-3′ linked DNA structures containing a core CpR dinucleotide, wherein the R is a modified guanosine (U.S. Pat. No. 7,276,489). In addition, specific chemical modifications have allowed the preparation of specific oligonucleotide analogs that generate distinct modulations of the immune response. In particular, structure activity relationship studies have allowed identification of synthetic motifs and novel DNA-based compounds that generate specific modulations of the immune response and these modulations are distinct from those generated by unmethylated CpG dinucleotides. (Kandimalla, E. et al. (2005) Proc. Natl. Acad. Sci. USA 102:6925-6930; Kandimalla, E. et al. (2003) Proc. Nat. Acad. Sci. USA 100:14303-14308; Cong, Y. et al. (2003) Biochem Biophys Res. Commun. 310:1133-1139; Kandimalla, E. et al. (2003) Biochem. Biophys. Res. Commun. 306:948-953; Kandimalla, E. et al. (2003) Nucleic Acids Res. 31:2393-2400; Yu, D. et al. (2003) Bioorg. Med. Chem. 11:459-464; Bhagat, L. et al. (2003) Biochem. Biophys. Res. Commun. 300:853-861; Yu, D. et al. (2002) Nucleic Acids Res. 30:4460-4469; Yu, D. et al. (2002) J. Med. Chem. 45:4540-4548. Yu, D. et al. (2002) Biochem. Biophys. Res. Commun. 297:83-90; Kandimalla. E. et al. (2002) Bioconjug. Chem. 13:966-974; Yu, D. et al. (2002) Nucleic Acids Res. 30:1613-1619; Yu, D. et al. (2001) Bioorg. Med. Chem. 9:2803-2808; Yu, D. et al. (2001) Bioorg. Med. Chem. Lett. 11:2263-2267; Kandimalla, E. et al. (2001) Bioorg. Med. Chem. 9:807-813; Yu, D. et al. (2000) Bioorg. Med. Chem. Lett. 10:2585-2588; and Putta, M. et al. (2006) Nucleic Acids Res. 34:3231-3238).

Different disease states or conditions may optimally be treated by different profiles of cytokines and/or chemokines. Accordingly, there exists a need in the field for new oligonucleotide analog compounds to provide such “custom-tuned” responses. In particular, there exists a need in the field for TLR9 agonists with specific and unique chemical modifications, which provide distinctive immune response activation profiles.

### BRIEF SUMMARY OF THE INVENTION

The inventors have surprisingly discovered that uniquely modifying the nucleic acid sequence flanking the core CpR dinucleotide, the linkages between nucleotides or the linkers connecting the oligonucleotides produces novel agonists of TLR9 that generate distinct in vitro and in vivo cytokine and chemokine profiles. This ability to “custom-tune” the cytokine and chemokine response to a CpR containing oligonucleotide provides the ability to prevent and/or treat various disease conditions in a disease-specific and even a patient-specific manner.

Thus, the invention provides, among other things, novel oligonucleotide-based compounds that individually provide distinct immune response profiles through their interactions as agonists with TLR9. The TLR9 agonists according to the invention are characterized by specific and unique chemical modifications, which provide their distinctive immune response activation profiles.

The TLR9 agonists according to the invention induce immune responses in various cell types and in various in vitro and in vivo experimental models, with each agonist providing a distinct immune response profile. The TLR9 agonists according to the invention are useful in the prevention and/or treatment of various diseases, either alone, in combination with or co-administered with other drugs, or as adjuvants for antigens used as vaccines. They are also useful as tools to study the immune system, as well as to compare the immune systems of various animal species, such as humans and mice.

Thus, in a first aspect, the invention provides oligonucleotide-based agonists of TLR9 (“a compound”).

In a second aspect, the invention provides pharmaceutical formulations comprising an oligonucleotide-based TLR9 agonist according to the invention and a pharmaceutically acceptable carrier.

In a third aspect, the invention provides a vaccine. Vaccines according to this aspect comprise a pharmaceutical formulation according to the invention and further comprise an antigen.

In a fourth aspect, the invention provides methods for generating a TLR9-mediated immune response in a subject, particularly a human, such methods comprising administering to the subject a compound, pharmaceutical formulation or vaccine according to the invention.

In a fifth aspect, the invention provides methods for therapeutically treating a patient having a disease or disorder, such methods comprising administering to the patient a compound, pharmaceutical formulation or vaccine according to the invention.

In a sixth aspect, the invention provides methods for preventing a disease or disorder, such methods comprising administering to a patient at risk of developing the disease or disorder a compound, pharmaceutical formulation or vaccine according to the invention.

In a seventh aspect, the invention provides a method for sensitizing cancer cells to ionizing radiation. The method according to this aspect of the invention comprises administering to a patient, a compound, pharmaceutical formulation or vaccine according to the invention and treating the patient with ionizing radiation.

### BRIEF DESCRIPTION OF THE DRAWINGS

FIG. 1 is a synthetic scheme for the synthesis of immune modulatory compounds of the invention. DMTr=4,4′-dimethoxytrityl; CE=cyanoethyl. All immune modulatory compounds of the invention were synthesized according to Example 1.

FIGS. 2A and 2B depict NF-kB activity in HEK293 cells expressing TLR9 that were cultured, treated and analyzed according to Example 2 below. Briefly, the HEK293 cells were stimulated with 0 (PBS/Media), 0.1, 0.3, 1.0, 3.0, or 10.0 μg/ml of immune modulatory oligonucleotides according to the invention for 18 hours, and the levels of NF-κB were determined using SEAP (secreted form of human embryonic alkaline phosphatase) assay. FIGS. 2A and 2B more generally demonstrate that administration of immune modulatory oligonucleotides containing novel bases, linkers, and/or unique modifications according to the invention generates distinct TLR9 activation profiles.

FIGS. 3A and 3B depict cytokine and chemokine concentrations from human PBMCs that were isolated, cultured, treated and analyzed according to Example 3 below. Briefly, the PBMCs were isolated from freshly obtained healthy human volunteer's blood and cultured with 0 (PBS), 0.1, 0.3, 1.0, 3.0, or 10.0 μg/ml of immune modulatory oligonucleotides according to the invention for 24 hours. Supernatants were collected and analyzed by Luminex multiplex assay for cytokine and chemokine levels. FIGS. 3A and 3B more generally demonstrate that administration of immune modulatory oligonucleotides containing novel bases, linkers, and/or unique modifications according to the invention generates distinct cytokine and chemokine profiles.

FIGS. 4A and 4B depict IFN-γ concentrations from human plasmacytoid dendritic cells (pDCs) that were isolated, cultured, treated and analyzed according to Example 3 below. Briefly, the pDCs were isolated from freshly obtained healthy human volunteer's blood PBMCs and cultured with 1 μg/ml of immune modulatory oligonucleotides according to the invention for 24 hr. Supernatants were collected and analyzed by Luminex multiplex assay for cytokine and chemokine levels. FIGS. 4A and 4B more generally demonstrate that administration of immune modulatory oligonucleotides containing novel bases, linkers, and/or unique modifications according to the invention generates distinct cytokine and chemokine profiles.

FIGS. 5A and 5B depict human B-cell proliferation induced by immune modulatory oligonucleotides according to the invention. The human B-cells were isolated, cultured, treated and analyzed according to Example 4 below. Briefly, the Human B cells isolated from freshly obtained healthy human volunteer's PBMCs were cultured with 0, 0.1, 0.3, 1.0, 3.0 or 10.0 μg/ml of immune modulatory oligonucleotides according to the invention for 68 hours and pulsed with ³H-thymidine for 6-8 hours. ³H-Thymidine uptake was determined using a liquid scintillation counter. FIGS. 5A and 5B more generally demonstrate that administration of immune modulatory oligonucleotides containing novel bases, linkers, and/or unique modifications according to the invention generates distinct cell proliferation profiles, which vary with the base composition, unique modification, and the amount of the oligonucleotide administered.

FIGS. 6A and 6B depict serum IL-12 induction in C57BL/6 mice that were treated according to Example 5 below. Briefly, 2 hours after the mice were injected subcutaneously with 1 mg/kg dosage of immune modulatory oligonucleotides according to the invention, serum was collected and analyzed by Luminex multiplex assay for cytokine and chemokine levels. FIGS. 6A and 6B more generally demonstrate that in vivo administration of immune modulatory oligonucleotides containing novel bases, linkers, and/or unique modifications according to the invention generates distinct TLR9 activation profiles, which will find application in a variety of diseases.

### DETAILED DESCRIPTION OF THE PREFERRED EMBODIMENTS

The invention provides novel oligonucleotide-based compounds that individually provide distinct immune response profiles through their interactions as agonists with TLR9. The TLR9 agonists according to the invention are characterized by unique chemical modifications, which provide their distinct immune response activation profiles.

The TLR9 agonists according to the invention induce immune responses in various cell types and in various in vivo and in vitro experimental models, with each agonist providing a distinct immune response profile. As such, they are useful as tools to study the immune system, as well as to compare the immune systems of various animal species, such as humans and mice. The TLR9 agonists according to the invention are also useful in the prevention and/or treatment of various diseases, either alone, in combination with or co-administered with other drugs, or as adjuvants for antigens used as vaccines.

The objects of the present invention, the various features thereof, as well as the invention itself may be more fully understood from the following description, when read together with the accompanying drawings in which the following terms have the ascribed meaning.

The term “2′-substituted nucleoside” or “2′-substituted arabinoside” generally includes nucleosides or arabinonucleosides in which the hydroxyl group at the 2′ position of a pentose or arabinose moiety is substituted to produce a 2′-substituted or 2′-O-substituted ribonucleoside. In certain embodiments, such substitution is with a lower hydrocarbyl group containing 1-6 saturated or unsaturated carbon atoms, with a halogen atom, or with an aryl group having 6-10 carbon atoms, wherein such hydrocarbyl, or aryl group may be unsubstituted or may be substituted, e.g., with halo, hydroxy, trifluoromethyl, cyano, nitro, acyl, acyloxy, alkoxy, carboxyl, carboalkoxy, or amino groups. Examples of 2′-O-substituted ribonucleosides or 2′-O-substituted-arabinosides include, without limitation 2′-amino, 2′-fluoro, 2′-allyl, 2′-O-alkyl and 2′-propargyl ribonucleosides or arabinosides, 2′-O-methylribonucleosides or 2′-O-methylarabinosides and 2′-O-methoxyethoxyribonucleosides or 2′-O-methoxyethoxyarabinosides.

The term “3′”, when used directionally, generally refers to a region or position in a polynucleotide or oligonucleotide 3′ (toward the 3′ position of the oligonucleotide) from another region or position in the same polynucleotide or oligonucleotide.

The term “5′”, when used directionally, generally refers to a region or position in a polynucleotide or oligonucleotide 5′ (toward the 5′ position of the oligonucleotide) from another region or position in the same polynucleotide or oligonucleotide.

The term “about” generally means that the exact number is not critical. Thus, the number of nucleoside residues in the oligonucleotides is not critical, and oligonucleotides having one or two fewer nucleoside residues, or from one to several additional nucleoside residues are contemplated as equivalents of each of the embodiments described above.

The term “airway inflammation” generally includes, without limitation, inflammation in the respiratory tract caused by allergens, including asthma.

The term “allergen” generally refers to an antigen or antigenic portion of a molecule, usually a protein, which elicits an allergic response upon exposure to a subject. Typically the subject is allergic to the allergen as indicated, for instance, by the wheal and flare test or any method known in the art. A molecule is said to be an allergen even if only a small subset of subjects exhibit an allergic (e.g., IgE) immune response upon exposure to the molecule.

The term “allergy” generally includes, without limitation, food allergies, respiratory allergies and skin allergies.

The term “antigen” generally refers to a substance that is recognized and selectively bound by an antibody or by a T cell or B cell antigen receptor and elicits a specific immune response. Antigens may include but are not limited to peptides, proteins, carbohydrates, lipids, nucleic acids and combinations thereof. Antigens may be natural or synthetic and generally induce an immune response that is specific for that antigen.

The term “cancer” generally refers to, without limitation, any malignant growth or tumor caused by abnormal or uncontrolled cell proliferation and/or division. Cancers may occur in humans and/or animals and may arise in any and all tissues. Treating a patient having cancer with the invention may include administration of a compound, pharmaceutical formulation or vaccine according to the invention such that the abnormal or uncontrolled cell proliferation and/or division is affected.

The term “carrier” generally encompasses any excipient, diluent, filler, salt, buffer, stabilizer, solubilizer, oil, lipid, lipid containing vesicle, microspheres, liposomal encapsulation, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990.

The terms “pharmaceutically acceptable” or “physiologically acceptable” generally refer to a material that does not interfere with the effectiveness of a compound according to the invention, and that is compatible with a biological system such as a cell, cell culture, tissue, or organism. Preferably, the biological system is a living organism, such as a vertebrate.

The terms “co-administration” or “co-administered” generally refers to the administration of at least two different substances sufficiently close in time to modulate an immune response. In some preferred embodiments, co-administration refers to simultaneous administration of at least two different substances.

The term a “pharmaceutically effective amount” generally refers to an amount sufficient to affect a desired biological effect, such as a beneficial result. Thus, a “pharmaceutically effective amount” will depend upon the context in which it is being administered. A pharmaceutically effective amount may be administered in one or more prophylactic or therapeutic administrations.

The term “in combination with” generally means administering a compound according to the invention and another agent useful for treating the disease or condition. Such administration may be done in any order, including simultaneous administration, as well as temporally spaced order from a few seconds up to several days apart or from hours to days apart, or hours apart. Such combination treatment may also include more than a single administration of the compound according to the invention and/or independently the other agent. The administration of the compound according to the invention and the other agent may be by the same or different routes.

The terms “subject” or “patient” generally refer to a mammal, such as a human. Mammals generally include, but are not limited to, humans, non-human primates, rats, mice, cats, dogs, horses, cattle, cows, pigs, sheep and rabbits.

The term “kinase inhibitor” generally refers to molecules that antagonize or inhibit phosphorylation-dependent cell signaling and/or growth pathways in a cell. Kinase inhibitors may be naturally occurring or synthetic and include small molecules that have the potential to be administered as oral therapeutics. Kinase inhibitors have the ability to rapidly and specifically inhibit the activation of the target kinase molecules. Protein kinases are attractive drug targets, in part because they regulate a wide variety of signaling and growth pathways and include many different proteins. As such, they have great potential in the treatment of diseases involving kinase signaling, including cancer, cardiovascular disease, inflammatory disorders, diabetes, macular degeneration and neurological disorders. Examples of kinase inhibitors include but are not limited to, erlotinib hydrochloride (Tarceva®), gefitinib (Iressa®), sorafenib tosylate (Nexavar®), sunititnib malate (Sutent®), dasatinib (Sprycel™), vandetanib (Zactima™), lapatinib (Tykerb™), temsirolimus (Toricel®), and imatinib mesylate (Gleevec®).

The term “mammal” is expressly intended to include warm blooded, vertebrate animals, including, without limitation, humans.

The term “modified nucleoside” generally refers to a nucleoside that includes a modified heterocyclic base, a modified sugar moiety, or any combination thereof. In some embodiments, the modified nucleoside is a non-natural pyrimidine or purine nucleoside, as herein described. For purposes of the invention, the terms “modified nucleoside”, “pyrimidine or purine analog” or “non-naturally occurring pyrimidine or purine” can be used interchangeably and refer to a nucleoside that includes a non-naturally occurring base and/or non-naturally occurring sugar moiety. For purposes of the invention, a base is considered to be non-natural if it is not guanine, cytosine, adenine, thymine or uracil.

The terms “modulation” or “modulatory” generally refer to change, such as an increase in a response or qualitative difference in a TLR9-mediated response.

The term “linker” generally refers to any moiety that can be attached to an oligonucleotide by way of covalent or non-covalent bonding through a sugar, a base, or the backbone. The linker can be used to attach two or more nucleosides or can be attached to the 5′ and/or 3′ terminal nucleotide in the oligonucleotide. In certain embodiments of the invention, such linker may be a non-nucleotidic linker.

The term “non-nucleotidic linker” generally refers to a chemical moiety other than a nucleotidic linkage that can be attached to an oligonucleotide by way of covalent or non-covalent bonding. Preferably such non-nucleotidic linker is from about 2 angstroms to about 200 angstroms in length, and may be either in a cis or trans orientation.

The term “nucleotidic linkage” generally refers to a chemical linkage to join two nucleosides through their sugars (e.g. 3′-3′, 2′-3′, 2′-5′, 3′-5′) consisting of a phosphorous atom and a charged, or neutral group (e.g., phosphodiester, phosphorothioate, phosphorodithioate, or alkylphosphonate) between adjacent nucleosides.

The term “oligonucleotide-based compound” refers to a polynucleoside formed from a plurality of linked nucleoside units. The nucleoside units may be part of or may be made part of viruses, bacteria, cell debris, siRNA or microRNA. Such oligonucleotides can also be obtained from existing nucleic acid sources, including genomic or cDNA, but are preferably produced by synthetic methods. In preferred embodiments each nucleoside unit includes a heterocyclic base and a pentofuranosyl, trehalose, arabinose, 2′-deoxy-2′-substituted nucleoside, 2′-deoxy-2′-substituted arabinose, 2′-O-substitutedarabinose or hexose sugar group. The nucleoside residues can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide-based compound” also encompasses polynucleosides having one or more stereospecific internucleoside linkage (e.g., (R_{P})- or (S_{P})-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain preferred embodiments, these internucleoside linkages may be phosphodiester, phosphorothioate or phosphorodithioate linkages, or combinations thereof.

The term “peptide” generally refers to amino acid oligomers that are of sufficient length and composition to affect a biological response, e.g., antibody production or cytokine activity whether or not the peptide is a hapten. The term “peptide” may include modified amino acids (whether or not naturally or non-naturally occurring), where such modifications include, but are not limited to, phosphorylation, glycosylation, pegylation, lipidization and methylation.

The term “TLR9 agonist” generally refers to an oligonucleotide-based compound that is able to enhance, induce or modulate an immune stimulation mediated by TLR9.

The term “treatment” generally refers to an approach intended to obtain a beneficial or desired result, which may include alleviation of symptoms, or delaying or ameliorating a disease progression.

In a first aspect, the invention provides oligonucleotide-based agonists of TLR9 (“a compound”). Certain TLR9 agonists according to the invention are shown in Table I below. In this table, the oligonucleotide-based TLR9 agonists have all phosphorothioate (PS) linkages, except where indicated. Those skilled in the art will recognize, however, that phosphodiester (PO) linkages, or a mixture of PS and PO linkages can be used. Except where indicated, all nucleotides are deoxyribonucleotides.

TABLE I

| SEQ | | | |
|---|---|---|---|
| ID | COMPOUND | | |
| NO | NO | Sequence and Modifications | COMPOUND Structure |
| 1 | 1 | 5′-TCG1TACG1TACG1-X1- | 5′-SEQ ID NO 1-3′-X1-3′- |
| | | G1CATG1CATG1CT-5′ | SEQ ID NO 1-5′ |
| 2 | 2 | 5′-TCG1TACoG1TACG1-X1- | 5′-SEQ ID NO 2-3′-X1-3′- |
| | | G1CATG1oCATG1CT-5′ | SEQ ID NO 2-5′ |
| 3 | 3 | 5′-TCG1TACoG1TACoG-X1- | 5′-SEQ ID NO 3-3′-X1-3′- |
| | | GoCATG1oCATG1CT-5′ | SEQ ID NO 3-5′ |
| 4 | 4 | 5′-TCG2TACoG2TACoG2-X1- | 5′-SEQ ID NO 4-3′-X1-3′- |
| | | G2oCATG2oCATG2CT-5′ | SEQ ID NO 4-5′ |
| 5 | 5 | 5′-TCG1TCoG1ACoGAT-X1- | 5′-SEQ ID NO 5-3′-X1-3′- |
| | | TAGoCAG1oCTG1CT-5′ | SEQ ID NO 5-5′ |
| 6 | 6 | 5′-TCG2TAoCoG2oTACG2-X1- | 5′-SEQ ID NO 6-3′-X1-3′- |
| | | G2CAToG2oCoATG2CT-5′ | SEQ ID NO 6-5′ |
| | 7 | Control TLR9 agonist 1 | |
| | 8 | Control TLR9 agonist 2 | |
| | 9 | Inactive oligonucleotide | |
| 10 | 10 | 5′-TCoG1oAACoG1TTCoG1o-X3- | 5′-SEQ ID NO 10-3′-X3-3′- |
| | | oG1oCTTG1oCAAoG1oCT-5′ | SEQ ID NO 10-5′ |
| 11 | 11 | 5′-ToCG1oAACoG1TTCoG1o-X3- | 5′-SEQ ID NO 11-3′-X3-3′- |
| | | oG1oCTTG1oCAAoG1CoT-5′ | SEQ ID NO 11-5′ |
| 12 | 12 | 5′-TCoG1oAACoG1TTCoG1o-m- | 5′-SEQ ID NO 12-3′-m-3′- |
| | | oG1oCTTG1oCAAoG1oCT-5′ | SEQ ID NO 12-5′ |
| 13 | 13 | 5′-TCoG2oAACoG2TTCoG2o-X3- | 5′-SEQ ID NO 13-3′-X3-3′- |
| | | oG2oCTTG2oCAAoG2oCT-5′ | SEQ ID NO 13-5′ |
| 14 | 14 | 5′-TCoG1oTCoG1ACoGATo-X1- | 5′-SEQ ID NO 14-3′-X1-3′- |
| | | oTAGoCAG1oCToG1oCT-5′ | SEQ ID NO 14-5′ |
| 15 | 15 | 5′-TCoG1oAACoG1TTCoGo-Z- | 5′-SEQ ID NO 15-3′-Z-3′- |
| | | oGoCTTG1oCAAoG1oCT-5′ | SEQ ID NO 15-5′ |
| 16 | 16 | 5′-ToCG1oAACoG1TTCoGo-Z- | 5′-SEQ ID NO 16-3′-Z-3′- |
| | | oGoCTTG1oCAAoG1CoT-5′ | SEQ ID NO 16-5′ |
| 17 | 17 | 5′-TCoG2oTACoG2TACoG2o-X1- | 5′-SEQ ID NO 17-3′-X1-3′- |
| | | oG2oCATG2oCAToG2oCT-5′ | SEQ ID NO 17-5′ |

G1 = 7-deaza-dG; G2 = arabinoG; G = 2′-O-methylribonucleotides; o = phosphodiester linkage; X1 = 1,2,4-butanetriol linker; X3 = 2-Hydroxymethyl-1,3-propanediol linker; m = cis,trans-1,3,5-Cyclohexanetriol linker; Z = 1,3,5-Pentanetriol linker.

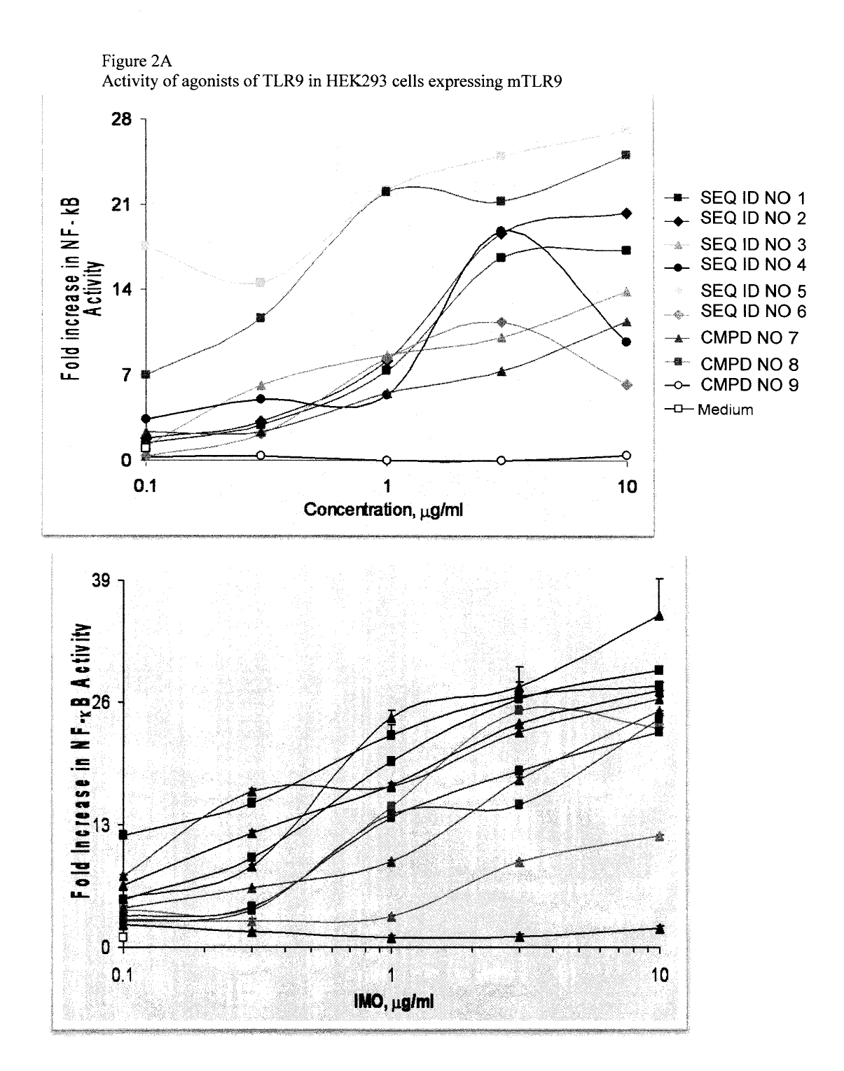

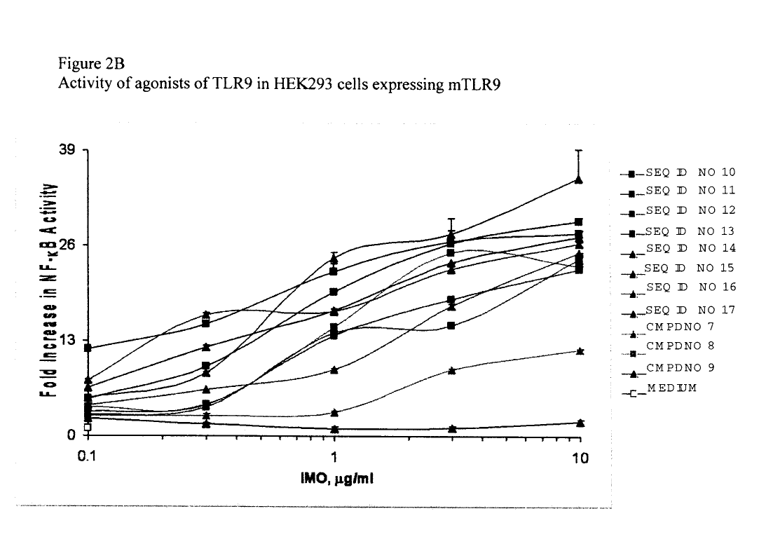

TLR9 agonists from Table I were tested for immune stimulatory activity in HEK293 cells expressing TLR9, as described in Example 2. The results shown in FIG. 2 demonstrate that specific chemical modifications to 3′-3′ linked oligonucleotides will alter their TLR9 mediated NF-kB activation profile 18 hours after administration. More generally, these data demonstrate that specific chemical modifications to 3′-3′ linked oligonucleotides can be used to increase or decrease NF-kB activity.

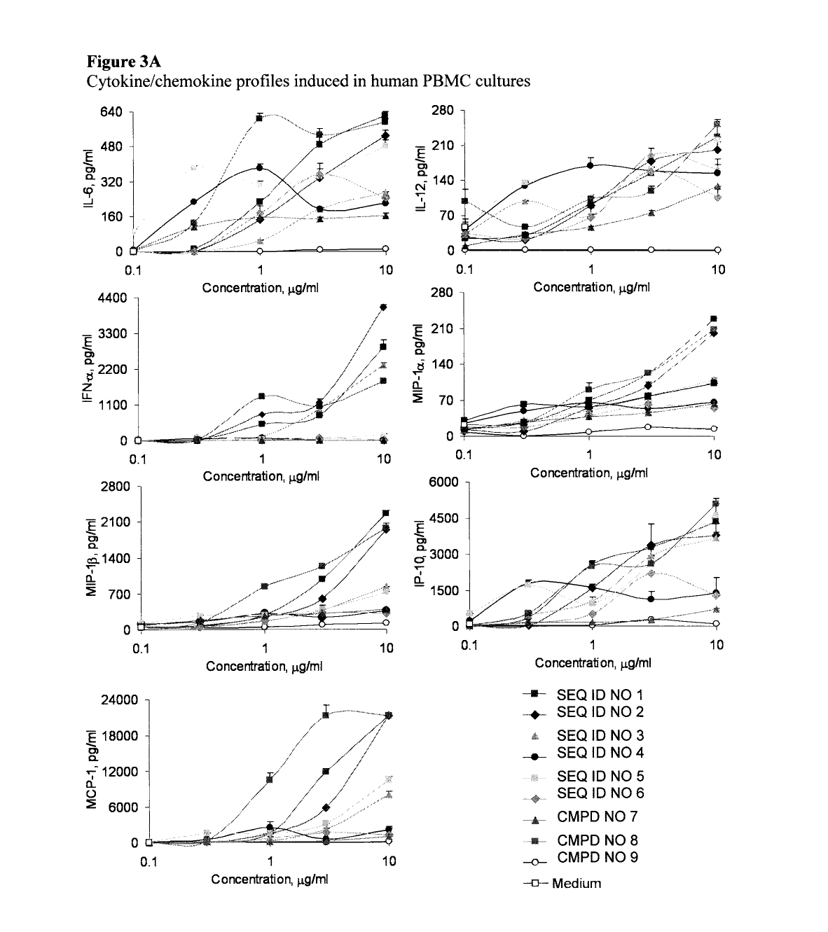

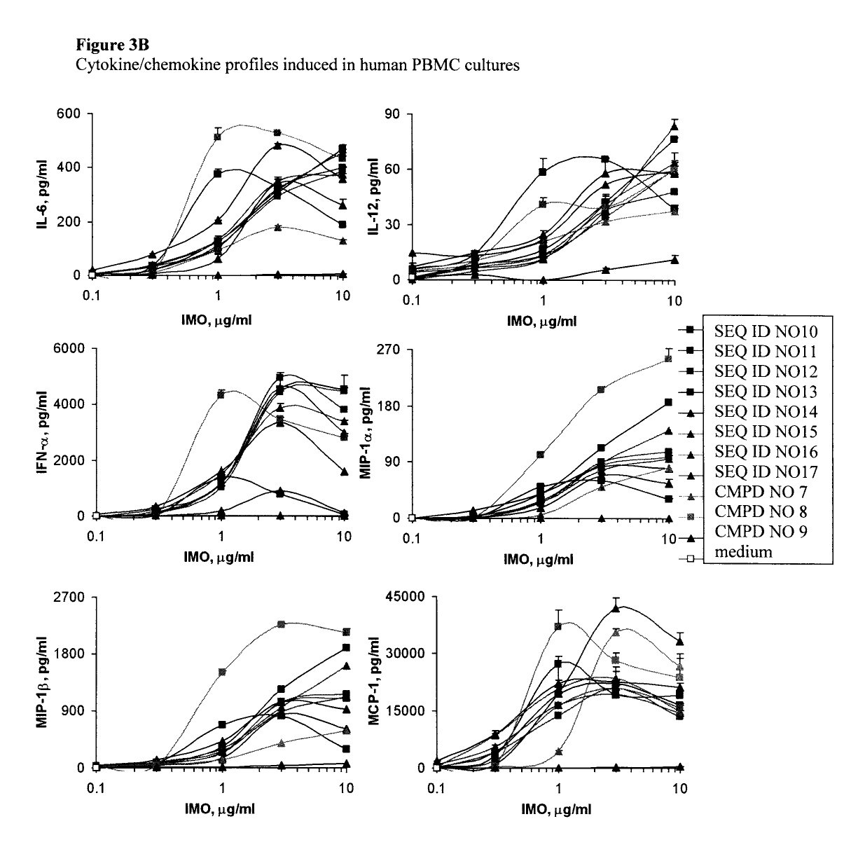

TLR9 agonists from Table I were tested for immune stimulatory activity in the human PBMC assay for IL-12, IL-6, IFN-α, IP-10, MIP-1α, MIP-1β, and MCP-1 as described in Example 3. The results shown in FIG. 3 demonstrate that specific chemical modifications to 3′-3′ linked oligonucleotides will alter their TLR9 mediated IL-12, IL-6, IFN-α, IP-10, MIP-1α, MIP-1β, and/or MCP-1 activation profile in human PBMCs. More generally, these data demonstrate that specific chemical modifications to 3′-3′ linked oligonucleotides can be used to increase or decrease IL-12, IL-10, IL-8, IL-6, IFN-α, IP-10, MIP-1α, MIP-1β, IL-1Rα, IL-2R, and MCP-1 activity.

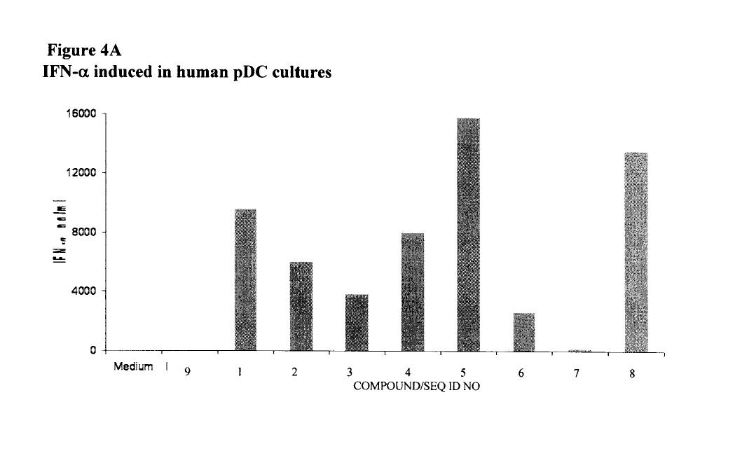

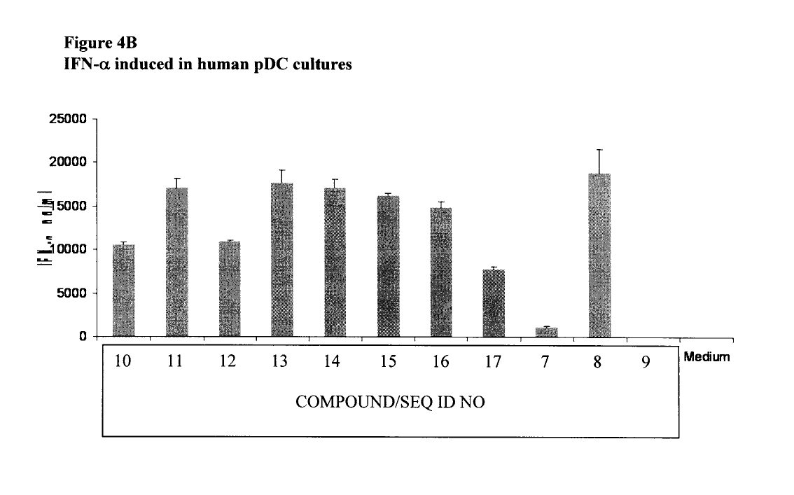

TLR9 agonists from Table I were tested for immune stimulatory activity in the human pDC assays for IL-12, IL-6, IFN-α, IP-10, MIP-1α, MIP-1β, and TNFα, as described in Example 3. The results shown in FIG. 4 demonstrate that specific chemical modifications to 3′-3′ linked oligonucleotides will alter their TLR9 mediated immune activation profile in human pDCs. More generally, these data demonstrate that specific chemical modifications to 3′-3′ linked oligonucleotides can be used to increase or decrease IL-12, IL-6, IFN-α, IP-10, MIP-1α, MIP-1β, and TNFα activity.

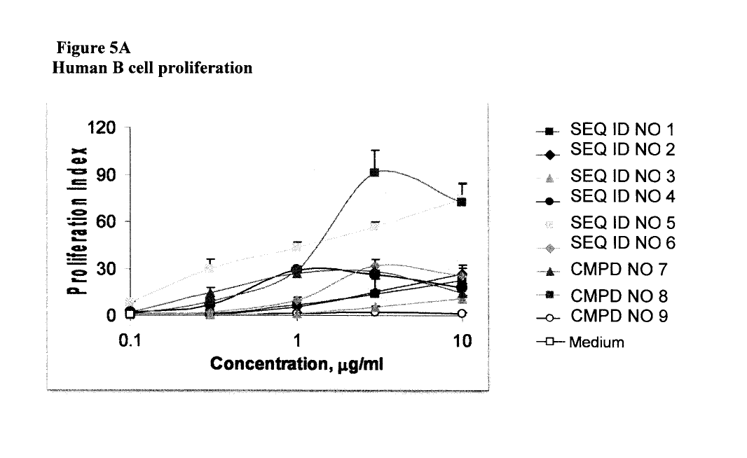

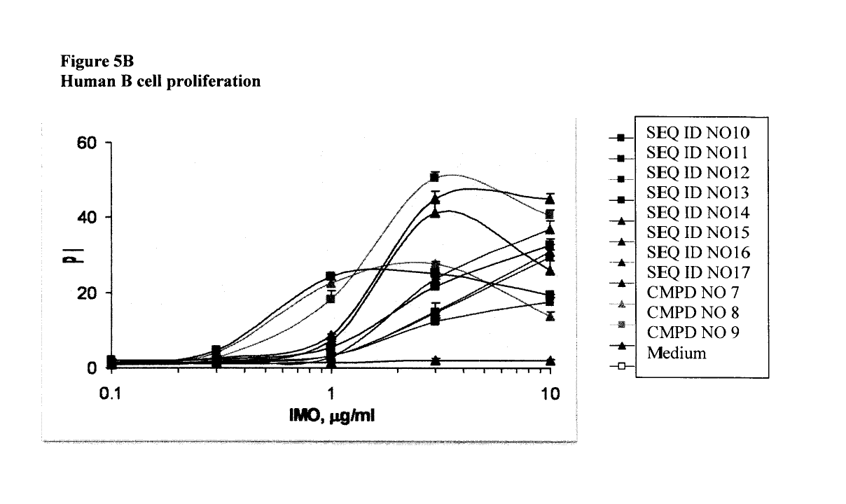

TLR9 agonists from Table I were tested for immune stimulatory activity in the human B-cell proliferation assay, as described in Example 4. The results shown in FIG. 5 demonstrate that specific chemical modifications to 3′-3′ linked oligonucleotides will alter their TLR9 mediated B-cell proliferation activity and that this activation profile may be dose dependent depending on the chemical modification. More generally, these data demonstrate that specific chemical modifications to 3′-3′ linked oligonucleotides can be used to regulate B-cell proliferation.

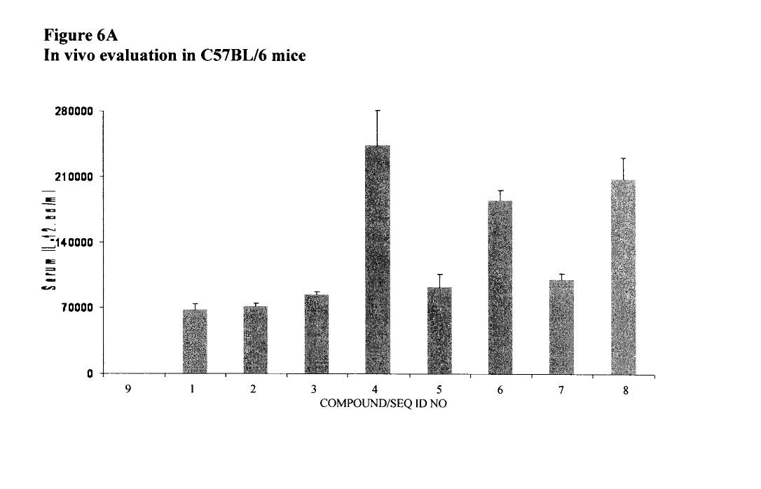

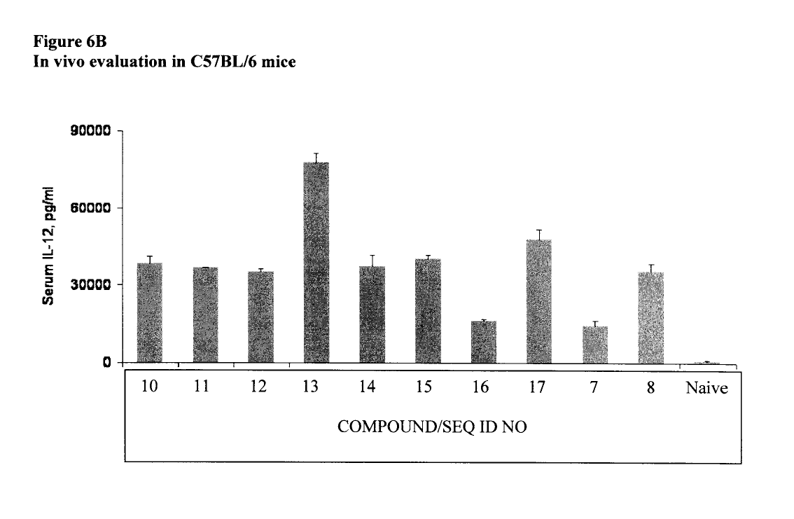

TLR9 agonists from Table I were tested for in vivo immune stimulatory activity in C57B1/6 mice, as described in Example 5. The results shown in FIG. 6 demonstrate that specific chemical modifications to 3′-3′ linked oligonucleotides will alter their in vivo TLR9 medicated immune activation profile in mouse models. More generally, these data demonstrate that specific chemical modifications to 3′-3′ linked oligonucleotides can alter in vivo cytokine and/or chemokine concentrations, which will find application in many diseases.

As described above, the invention provides, in a first aspect, oligonucleotide-based synthetic agonists of TLR9. Based upon certain chemical modifications to the base, sugar, linkage or linker, the agonists of TLR9 may possess increased stability when associated and/or duplexed with other of the TLR9 agonist molecules, while retaining an accessible 5′-end.

In some embodiments, the non-nucleotidic linker may include, but are not limited to, 1,2,4-Butanetriol, cis,trans-1,3,5-Cyclohexanetriol, 1,3,5-Pentanetriol or 2-Hydroxymethyl-1,3-propanediol.

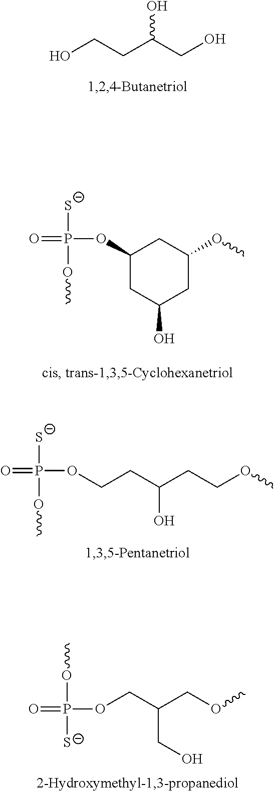

In a second aspect, the invention provides pharmaceutical formulations comprising an oligonucleotide-based TLR9 agonist (“a compound”) according to the invention and a pharmaceutically acceptable carrier.

The active compound is included in the pharmaceutically acceptable carrier or diluent in an amount sufficient to deliver to a patient a pharmaceutically effective amount without causing serious toxic effects in the patient treated. The effective dosage range of the pharmaceutically acceptable derivatives can be calculated based on the weight of the parent compound to be delivered, or by other means known to those skilled in the art. If the derivative exhibits activity in itself, the effective dosage can be estimated as above using the weight of the derivative, or by other means known to those skilled in the art.

In a third aspect, the invention provides a vaccine. Vaccines according to this aspect comprise a pharmaceutical formulation according to the invention, and further comprise an antigen. An antigen is a molecule that elicits a specific immune response. Such antigens include, without limitation, proteins, peptides, nucleic acids, carbohydrates, lipids and complexes or combinations of any of the same. Any such antigen may optionally be linked to an immunogenic protein or peptide, such as keyhole limpet hemocyanin (KLH), cholera toxin B subunit, or any other immunogenic carrier protein.

Vaccines according to the invention may further include any of a plethora of adjuvants, including, without limitation, Freund's complete adjuvant, Keyhole Limpet Hemocyanin (KLH), monophosphoryl lipid A (MPL), alum, and saponins, including QS-21, imiquimod, R848, TLR agonists or combinations thereof.

In a fourth aspect, the invention provides methods for generating a TLR9-mediated immune response in a subject, such methods comprising administering to the subject a compound, pharmaceutical formulation or vaccine according to the invention. In some embodiments, the subject is a human. In preferred embodiments, the compound, pharmaceutical formulation or vaccine is administered to a subject in need of immune stimulation. In certain preferred embodiments, the subject is a human.

In the methods according to this aspect of the invention, administration of a compound, pharmaceutical formulation or vaccine according to the invention can be by any suitable route, including, without limitation, parenteral, oral, intratumoral, sublingual, transdermal, topical, intranasal, aerosol, intraocular, intratracheal, intrarectal, mucosal, vaginal, by gene gun, dermal patch or in eye drop or mouthwash form. Administration of the compound, pharmaceutical formulation or vaccine can be carried out using known procedures at dosages and for periods of time effective to reduce symptoms or surrogate markers of the disease. When administered systemically, the compound, pharmaceutical formulation or vaccine is preferably administered at a sufficient dosage to attain a blood level of a compound according to the invention from about 0.0001 micromolar to about 10 micromolar. For localized administration, much lower concentrations than this may be effective, and much higher concentrations may be tolerated without serious toxic effects. Preferably, a total dosage of a compound according to the invention ranges from about 0.001 mg per patient per day to about 200 mg per kg body weight per day. It may be desirable to administer simultaneously, or sequentially a therapeutically effective amount of one or more of the therapeutic compositions of the invention to a subject as a single treatment episode.

In certain preferred embodiments, a compound, pharmaceutical formulation or vaccine according to the invention is co-administered or administered in combination with another agent, including without limitation chemotherapeutic agents, antibodies, cytotoxic agents, allergens, antibiotics, antisense oligonucleotides, siRNA molecules, aptamers, ribozymes, targeted therapeutics, kinase inhibitors, peptides, proteins, gene therapy vectors, DNA vaccines and/or adjuvants to enhance the specificity or magnitude of the immune response, radioisotopes, and ionizing radiation.

The methods according to this aspect of the invention are useful for the prophylactic or therapeutic treatment of human or animal disease. For example, the methods are useful for human adult and pediatric and veterinary vaccine applications. The methods are also useful for model studies of the immune system.

In a fifth aspect, the invention provides methods for therapeutically treating a patient having a disease or disorder, such methods comprising administering to the patient a compound, pharmaceutical formulation or vaccine according to the invention. In various embodiments, the disease or disorder to be treated is cancer, infectious disease, airway inflammation, inflammatory disorders, allergy, asthma or a disease caused by a pathogen or allergen. Pathogens include for example bacteria, parasites, fungi, viruses, viroids, and prions. Administration is carried out as described for the fourth aspect of the invention.

In a sixth aspect, the invention provides methods for preventing a disease or disorder, such methods comprising administering to a patient at risk for developing the disease or disorder, a compound, pharmaceutical formulation or vaccine according to the invention. In various embodiments, the disease or disorder to be prevented is cancer, airway inflammation, inflammatory disorders, infectious disease, allergy, asthma or a disease caused by a pathogen. Pathogens include, without limitation, bacteria, parasites, fungi, viruses, viroids, and prions. Administration is carried out as described for the fourth aspect of the invention. A patient at risk for developing a disease or disorder is generally a patient that has been or will be exposed to one or more etiological agent or causative condition of the disease or disorder and/or having a genetic predisposition for the disease or disorder.

In a seventh aspect, the invention provides a method for sensitizing cancer cells to ionizing radiation. The method according to this aspect of the invention comprises administering to a patient a compound, pharmaceutical formulation or vaccine according to the invention in combination with ionizing radiation. In certain preferred embodiments, the ionizing radiation is administered at about 1.56 Gy/min. In certain preferred embodiments, radiation therapy is administered as about 3 Gy of radiation either twice for one week, or four times for one week. In alternative embodiments a compound, pharmaceutical formulation or vaccine according to the invention is administered to the patient on a first day (Day 0) and radiation therapy is administered as about 3 Gy of radiation 2 days thereafter (Day 2), 4 days thereafter (Day 4), and 9 days thereafter (Day 9). In certain embodiments pre-treatment with compound, pharmaceutical formulation is from about 2 hours to about 6 hours prior to y-irradiation.

In any of the methods according to the invention, the compound, pharmaceutical formulation or vaccine according to the invention can be co-administered or administered in combination with any other agent useful for preventing or treating the disease or condition that does not abolish the immune stimulatory effect of the compound, pharmaceutical formulation or vaccine according to the invention. In any of the methods according to the invention, the other agent useful for preventing or treating the disease or condition includes, but is not limited to, vaccines, antigens, antibodies, cytotoxic agents, allergens, antibiotics, antisense oligonucleotides, siRNA molecules, aptamers, ribozymes, targeted therapeutics, TLR agonists, kinase inhibitors, peptides, proteins, gene therapy vectors, DNA vaccines and/or adjuvants to enhance the specificity or magnitude of the immune response, or co-stimulatory molecules such as cytokines chemokines, protein ligands, trans-activating factors, peptides and peptides comprising modified amino acids. For example, in the prevention and/or treatment of cancer, it is contemplated that the compound, pharmaceutical formulation or vaccine according to the invention may be co-administered or administered in combination with a chemotherapeutic compound, a monoclonal antibody, a radioisotope, or ionizing radiation. Preferred chemotherapeutic agents used in the method according to the invention include, without limitation Gemcitabine, methotrexate, vincristine, adriamycin, cisplatin, non-sugar containing chloroethylnitrosoureas, 5-fluorouracil, mitomycin C, bleomycin, doxorubicin, dacarbazine, paclitaxel, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin®/Topotecan, PKC412, Valspodar/PSC833, Novantrone®/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron®/strontium derivative, Temodal/Temozolomide, Evacet/liposomal doxorubicin, Yewtaxan/Paclitaxel, Taxol®/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT™ (Tegafur/Uracil), Ergamisol®/Levamisole, Eniluracil/776C85/5FU enhancer, Campto/Levamisole, Camptosar®/Irinotecan, Tumodex/Ralitrexed, Leustatin®/Cladribine, Paxex/Paclitaxel, Doxil®/liposomal doxorubicin, Caelyx/liposomal doxorubicin, Fludara®/Fludarabine, Pharmarubicin/Epirubicin, DepoCyt®/Cytarabine, ZD1839, LU 79553/Bis-Naphtalimide, LU 103793/Dolastain, Caetyx/liposomal doxorubicin, Gemzar®/Gemcitabine, ZD 0473(AnorMED®), YM 116, Iodine seeds, CDK4 and CDK2 inhibitors, PARP inhibitors, D4809/Dexifosamide, Ifex®/Ifosfamide, Mesnex®/Mesna, Vumon®/Teniposide, Paraplatin®/Carboplatin, Plantinol/cisplatin, Vepeside/Etoposide, ZD 9331, Taxotere®/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26) and Vindesine sulfate. Preferred monoclonal antibodies include, but are not limited to, Panorex® (Glaxo-Welcome), Rituxan® (IDEC/Genentech/Hoffman la Roche), Mylotarg® (Wyeth), Campath® (Millennium), Zevalin® (IDEC and Schering AG), Bexxar® (Corixa/GSK), Erbitux® (Imclone/BMS), Avastin® (Genentech) and Herceptin® (Genentech/Hoffman la Roche).

Alternatively, the agent useful for preventing or treating the disease or condition can include DNA vectors encoding for antigen or allergen. In these embodiments, the compound, pharmaceutical formulation or vaccine according to the invention can variously act as adjuvants and/or produce direct immunomodulatory effects.

The patents and publications cited herein reflect the level of knowledge in the art and are hereby incorporated by reference in their entirety. Any conflict between the teachings of these patents and publications and this specification shall be resolved in favor of the latter. Those skilled in the art will recognize, or be able to ascertain, using no more than routine experimentation, numerous equivalents to the specific substances and procedures described herein.

The following examples illustrate exemplary modes of making and practicing the present invention, but are not meant to limit the scope of the invention since alternative methods may be utilized to obtain similar results.

### Example 1

### Synthesis of Oligonucleotide-Based Compounds Containing Immune Stimulatory Moieties

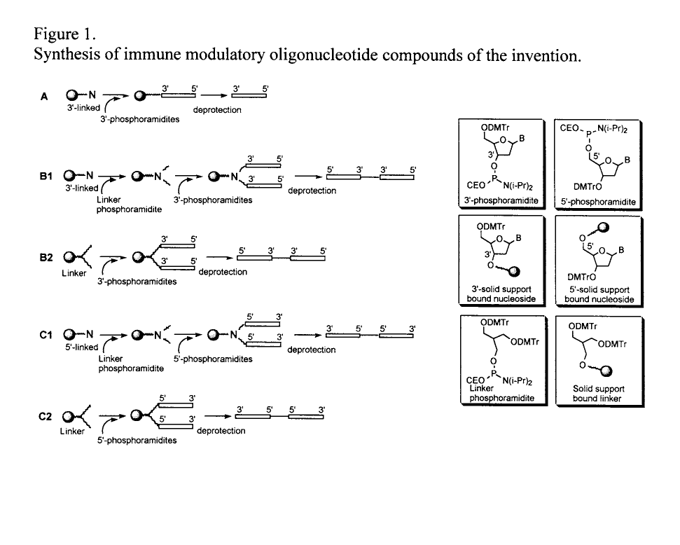

Chemical entities according to the invention were synthesized on a 1 μmol to 0.1 mM scale using an automated DNA synthesizer (OligoPilot II, AKTA, (Amersham) and/or Expedite 8909 (Applied Biosystem)), following the parallel synthesis procedure outlined in FIG. 1.

5′-DMT dA, dG, dC and T phosphoramidites were purchased from Proligo (Boulder, Colo.). 5′-DMT 7-deaza-dG and araG phosphoramidites were obtained from Chemgenes (Wilmington, Mass.). DiDMT-glycerol linker solid support was obtained from Chemgenes. 1-(2′-deoxy-β-D-ribofuranosyl)-2-oxo-7-deaza-8-methyl-purine amidite was obtained from Glen Research (Sterling, Va.), 2′-O-methylribonuncleoside amidites were obtained from Promega (Obispo, Calif.). All compounds according to the invention were phosphorothioate backbone modified.

All nucleoside phosphoramidites were characterized by ³¹P and ¹H NMR spectra. Modified nucleosides were incorporated at specific sites using normal coupling cycles recommended by the supplier. After synthesis, compounds were deprotected using concentrated ammonium hydroxide and purified by reverse phase HPLC, detritylation, followed by dialysis. Purified compounds as sodium salt form were lyophilized prior to use. Purity was tested by CGE and MALDI-TOF MS. Endotoxin levels were determined by LAL test and were below 1.0 EU/mg.

### Example 2

### Cell Culture Conditions and Reagents

HEK293 or HEK293XL cells expressing mouse TLR9 (Invivogen, San Diego, Calif.) were cultured in 48-well plates in 250 μl/well DMEM supplemented with 10% heat-inactivated FBS in a 5% CO₂ incubator. At 80% confluence, cultures were transiently transfected with 400 ng/ml of SEAP (secreted form of human embryonic alkaline phosphatase) reporter plasmid (pNifty2-Seap) (Invivogen) in the presence of 4 μl/ml of lipofectamine (Invitrogen, Carlsbad, Calif.) in culture medium. Plasmid DNA and lipofectamine were diluted separately in serum-free medium and incubated at room temperature for 5 minutes. After incubation, the diluted DNA and lipofectamine were mixed and the mixtures were incubated at room temperature for 20 minutes. Aliquots of 25 μl of the DNA/lipofectamine mixture containing 100 ng of plasmid DNA and 1 μl of lipofectamine were added to each well of the cell culture plate, and the cultures were continued for 4 hours.

Cytokine Induction by Immune Modulatory Compounds from Table I in HEK293 Cells Expressing Mouse TLR9

After transfection, medium was replaced with fresh culture medium, individual immune modulatory compounds from Table I were added to the cultures at concentrations of 0, 0.1, 0.3, 1.0, 3.0, or 10.0 μg/ml, and the cultures were continued for 18 hours. At the end of compounds treatment, the levels of NF-κB were determined using SEAP (secreted form of human embryonic alkaline phosphatase) assay according to the manufacturer's protocol (Invivogen). Briefly, 30 μl of culture supernatant was taken from each treatment and incubated with p-nitrophynyl phosphate substrate and the yellow color generated was measured by a plate reader at 405 nm (Putta M R et al, Nucleic Acids Res., 2006, 34:3231-8).

### Example 3

### Cytokine Induction by Immune Modulatory Compounds from Table I in Human PBMCs, pDCs, and Mouse Splenocytes

### Human PBMC Isolation

Peripheral blood mononuclear cells (PBMCs) from freshly drawn healthy volunteer blood (CBR Laboratories, Boston, Mass.) were isolated by Ficoll density gradient centrifugation method (Histopaque-1077, Sigma).

### Human pDC Isolation

Human plasmacytoid dendritic cells (pDCs) were isolated from freshly obtained healthy human volunteer's blood PBMCs by positive selection using the BDCA4 cell isolation kits (Miltenyi Biotec) according to the manufacturer's instructions.

Human PBMCs were plated in 48-well plates using 5×10⁶ cells/ml. Human pDCs were plated in 96-well dishes using 1×10⁶ cells/ml. Individual immune modulatory compounds from Table I were dissolved in DPBS (pH 7.4; Mediatech) were added to the cell cultures at doses of 0, 0.1, 0.3, 1.0, 3.0, or 10.0 μg/ml. The cells were then incubated at 37° C. for 24 hours and the supernatants were collected for luminex multiplex or ELISA assays. In certain experiments, the levels of IFN-α, IL-6, and/or IL-12 were measured by sandwich ELISA. The required reagents, including cytokine antibodies and standards, were purchased from PharMingen.

### Cytokine Luminex Multiplex

In certain experiments, the levels of IL-1Rα, IL-6, IL-10, IL-12, IFN-α, IFN-γ, MIP-1α, MIP-β, MCP-1, and IL-12p40p70 in culture supernatants were measured by Luminex multiplex assays, which were performed using Biosource human multiplex cytokine assay kits on Luminex 100 instrument and the data were analyzed using StarStation software supplied by Applied Cytometry Systems (Sacramento, Calif.).

### Activation of Human Immune Cells.

Human plasmacytoid dendritic cells (pDCs) were isolated from freshly obtained healthy human blood PBMCs and cultured with 50 μg/ml of individual immune modulatory compounds from Table I or control for 24 hr. Cells were stained with fluorescently-conjugated Abs (CD123, CD80, CD86) and data were collected on an FC500 MPL cytometer. Mean fluorescence intensity of CD80 and CD86 on CD123⁺ cells was analyzed using FlowJo software and is expressed as fold change over PBS control.

Human myeloid dendritic cells (mDC) were isolated from freshly obtained healthy human blood PBMCs and cultured with 50 μg/ml of individual immune modulatory compounds from Table I or control for 24 hr. Cells were stained with fluorescently-conjugated Abs (CD11c, CD80, CD40) and data were collected on an FC500 MPL cytometer. Mean fluorescence intensity of CD80 and CD40 on CD11c⁺ cells was analyzed using FlowJo software and is expressed as fold change over PBS control.

### Example 4

### Human B Cell Proliferation in the Presence of Immune Modulatory Compounds from Table I

Human B cells were isolated from PBMCs by positive selection using the CD19 Cell Isolation Kit (Miltenyi Biotec, Auburn, Calif.) according to the manufacturer's instructions.

The culture medium used for the assay consisted of RPMI 1640 medium supplemented with 1.5 mM glutamine, 1 mM sodium pyruvate, 0.1 mM non-essential amino acids, 50 μM 2-mercaptoethanol, 100 IU/ml penicillin-streptomycin mix and 10% heat-inactivated fetal bovine serum.

A total of 0.5×10⁶ B cells per ml (i.e. 1×10⁵/200 μl/well) were stimulated in 96 well flat bottom plates with different concentrations of individual immune modulatory compounds from Table I in triplicate for a total period of 68 hours. After 68 hours, cells were pulsed with 0.75 μCi of [³H]-thymidine (1 Ci=37 GBq; Perkin Elmer Life Sciences) in 20 μl RPMI 1640 medium (no serum) per well and harvested 6-8 hours later. The plates were then harvested using a cell harvester and radioactive incorporation was determined using standard liquid scintillation technique. In some cases the corresponding [³H]-T (cpm) was converted into a proliferation index and reported as such.

### Example 5

### In Vivo Cytokine Secretion in Mouse Model Treated with TLR9 Agonist Compounds

C57BL/6 mice, 5-6 weeks old, were obtained from Taconic Farms, Germantown, N.Y. and maintained in accordance with Idera Pharmaceutical's IACUC approved animal protocols. Mice (n=3) were injected subcutaneously with individual immune modulatory compounds from Table I at 0.25 or 1.0 mg/kg (single dose). Serum was collected by retro-orbital bleeding 2 hours after immune modulatory compound administration and IL-12, IL-10, IL-6, IP-10, KC, MCP1, MIG, MIP-1α and TNF-α concentrations were determined by sandwich ELISA or Luminex multiplex assays. The results are shown in FIG. 6 and demonstrate that in vivo administration of immune modulatory compounds containing novel chemical compositions generates unique cytokine and chemokine profiles. All reagents, including cytokine and chemokine antibodies and standards were purchased from PharMingen. (San Diego, Calif.).

### Example 6

### Radiosensitization by Oligonucleotides

The models of LNCaP, PC-3, MCF-7, MDA-MB-468, or PANC-1 xenografts are established using established procedures (see e.g., Wang, H., Nan, L., Yu, D., Agrawal, S. and Zhang, R., Clin. Cancer Res., 7: 3613-3624 (2001), Wang, H., Wang, S., Nan, L., Yu, D., Agrawal, S. and Zhang, R., Intl. J. Oncol., 20: 745-752 (2002), Prasad, G., Wang, H., Agrawal, S. and Zhang, R. Anticancer Res., 22: 107-116 (2002), Wang, H., Nan, L., Yu, D., Lindsey, J. R., Agrawal, S. and Zhang, R., Mol. Med., 8: 184-198 (2002), Wang, H., Yu, D., Agrawal, S. and Zhang, R., Prostate, 54: 194-205 (2003)). Briefly, MCF-7, MDA-MB-468, and P ANC-1 models use female athymic nude mice (nu/nu, 4-6 weeks old). For the LNCaP in vivo model male SCID mice (4-6 weeks old) are used and male athymic nude mice (nu/nu, 4-6 weeks old) are used for the PC-3 model. All mice are obtained from Frederick Cancer Research and Development Center (Frederick, Md., USA). Cultured cells are washed with serum-free media and resuspended in the same medium. This suspension (5×10⁶ cells, 0.2 ml mouse) is then injected into the left inguinal area of the mice. BMMx is combined with this suspension prior to injection at a ratio of 1:1 (LNCaP) or 1:5 (PC-3, MCF-7, MDA-MB-468, and P ANC-1). The mice are monitored by general clinical observation as well as by body weight and tumor growth. Tumor growth is recorded with the use of calipers, by measuring the long and short diameters of the tumor. Tumor mass (in g) is calculated using the formula 1/2a×b2, where ‘a’ and ‘b’ are the long and short diameters (in cm), respectively.

Mice bearing LNCaP, PC-3, MCF-7, MDA-MB-468, or P ANC-1 xenografts are randomly divided into multiple treatment groups in addition to a control group (5 mice/group). Immunomer or inactive immunomer control dissolved in sterile physiological saline (0.9% NaCl) is given by intraperitoneal (i.p.) injection (volume, 5 ˜1/g body weight) at a dose of 25 mg/kg, 5 times/week. Mice in radiation groups are first anesthetized with a 70-100 microliter mixture ofketamine (20 mg/ml) and xylazine (20 mg/ml) at a 1:6.7 ratio and then placed under a specially designed lead shield so that only the tumors are exposed to the radiation beam. y-Irradiation is administered by a 6000 Picker unit irradiator (JL Shepard Co., Glendale, Calif., USA) [1.56 Gy/min]. Animals receive 3 Gy of radiation either twice for one week (LNCaP), four times for one week (PC-3), or three times on Days 2, 4, and 9 (MCF-7, MDA-MB-468 and P ANC-1). Mice in oligo/radiation combination groups are pre-treated with oligo 4 h prior to irradiation.

It is expected that the inactive immunomer control will have no effect on tumor growth, similar to controls treated with saline. Immunomer compound alone will show anti-tumor activity, as will radiation alone. Immunomer compound in combination with radiation will show improved or synergistic anti-tumor activity compared with either treatment alone.

1. An immune modulatory compound comprising an oligonucleotide-based compound having a sequence selected from SEQ ID NOs 2, 3, 4, 5, 6, 10, 11, 12, 13, 14, 15, 16, and 17.

2. A composition comprising the immune modulatory compound of claim 1 and a physiologically acceptable carrier.

3. A method for generating an immune response in a subject, the method comprising administering to the subject a compound according to claim 1 in a pharmaceutically effective amount.

4. A method for therapeutically treating a subject having a disease or disorder where modulating an immune response would be beneficial, such method comprising administering to the subject a compound according to claim 1 in a pharmaceutically effective amount.

5. The method of claim 4 where in the disease or disorder is cancer, airway inflammation, inflammatory disorder, infectious disease, skin disorder, allergy, asthma or a disease caused by a pathogen or allergen.

6. The method according to claim 4, further comprising administering one or more chemotherapeutic compounds, targeted therapeutic agents, vaccines, adjuvants, antigens, antibodies, cytotoxic agents, allergens, antibiotics, antisense oligonucleotides, siRNA molecules, aptamers, riboxymes, TLR agonists, kinase inhibitors, peptides, proteins, DNA vaccines, adjuvants, co-stimulatory molecules, radioisotopes or ionizing radiation, or combinations thereof.

7. The method according to claim 6, wherein the antibody is selected from Panorex® (Glaxo-Welcome), Rituxan® (IDEC/Genentech/Hoffman la Roche), Mylotarg® (Wyeth), Campath® (Millennium), Zevalin® (IDEC and Schering AG), Bexxar® (Corixa/GSK), Erbitux® (Imclone/BMS), Avastin® (Genentech) and Herceptin® (Genentech/Hoffman la Roche).

8. The method according to claim 6 wherein the chemotherapeutic compound is selected from Gemcitabine, methotrexate, vincristine, adriamycin, cisplatin, non-sugar containing chloroethylnitrosoureas, 5-fluorouracil, mitomycin C, bleomycin, doxorubicin, dacarbazine, paclitaxel, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin®/Topotecan, PKC412, Valspodar/PSC833, Novantrone®/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron®/strontium derivative, Temodal/Temozolomide, Evacet/liposomal doxorubicin, Yewtaxan/Paclitaxel, Taxol®/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT™ (Tegafur/Uracil), Ergamisol®/Levamisole, Eniluracil/776C85/5FU enhancer, Campto/Levamisole, Camptosar®/Irinotecan, Tumodex/Ralitrexed, Leustatin®/Cladribine, Paxex/Paclitaxel, Doxil®/liposomal doxorubicin, Caelyx/liposomal doxorubicin, Fludara®/Fludarabine, Pharmarubicin/Epirubicin, DepoCyt®/Cytarabine, ZD1839, LU 79553/Bis-Naphtalimide, LU 103793/Dolastain, Caetyx/liposomal doxorubicin, Gemzar®/Gemcitabine, ZD 0473(AnorMED®), YM 116, Iodine seeds, CDK4 and CDK2 inhibitors, PARP inhibitors, D4809/Dexifosamide, Ifex®/Ifosfamide, Mesnex®/Mesna, Vumon®/Teniposide, Paraplatin®/Carboplatin, Plantinol/cisplatin, Vepeside/Etoposide, ZD 9331, Taxotere®/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26) and Vindesine sulfate.

9. A method for prophylactically treating a subject at risk for developing a disease or disorder where modulating an immune response would be beneficial, such method comprising administering to the subject a compound according to claim 1 in a pharmaceutically effective amount.

10. The method of claim 9 wherein the disease or disorder is cancer, airway inflammation, inflammatory disorders, infectious disease, skin disorders, allergy, asthma or a disease caused by a pathogen or allergen in the subject.

11. The method according to claim 9, further comprising administering one or more chemotherapeutic compounds, targeted therapeutic agents, vaccines, adjuvants, antigens, antibodies, cytotoxic agents, allergens, antibiotics, antisense oligonucleotides, siRNA molecules, aptamers, riboxymes, TLR agonists, kinase inhibitors, peptides, proteins, DNA vaccines, adjuvants, co-stimulatory molecules, radioisotopes or ionizing radiation, or combinations thereof.

12. The method according to claim 11, wherein the antibody is selected from Panorex® (Glaxo-Welcome), Rituxan® (IDEC/Genentech/Hoffman la Roche), Mylotarg® (Wyeth), Campath® (Millennium), Zevalin® (IDEC and Schering AG), Bexxar® (Corixa/GSK), Erbitux® (Imclone/BMS), Avastin® (Genentech) and Herceptin® (Genentech/Hoffman la Roche).

13. The method according to claim 11 wherein the chemotherapeutic compound is selected from Gemcitabine, methotrexate, vincristine, adriamycin, cisplatin, non-sugar containing chloroethylnitrosoureas, 5-fluorouracil, mitomycin C, bleomycin, doxorubicin, dacarbazine, paclitaxel, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin®/Topotecan, PKC412, Valspodar/PSC833, Novantrone®/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron®/strontium derivative, Temodal/Temozolomide, Evacet/liposomal doxorubicin, Yewtaxan/Paclitaxel, Taxol®/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT™ (Tegafur/Uracil), Ergamisol®/Levamisole, Eniluracil/776C85/5FU enhancer, Campto/Levamisole, Camptosar®/Irinotecan, Tumodex/Ralitrexed, Leustatin®/Cladribine, Paxex/Paclitaxel, Doxil®/liposomal doxorubicin, Caelyx/liposomal doxorubicin, Fludara®/Fludarabine, Pharmarubicin/Epirubicin, DepoCyt®/Cytarabine, ZD1839, LU 79553/Bis-Naphtalimide, LU 103793/Dolastain, Caetyx/liposomal doxorubicin, Gemzar®/Gemcitabine, ZD 0473(AnorMED®), YM 116, Iodine seeds, CDK4 and CDK2 inhibitors, PARP inhibitors, D4809/Dexifosamide, Ifex®/Ifosfamide, Mesnex®/Mesna, Vumon®/Teniposide, Paraplatin®/Carboplatin, Plantinol/cisplatin, Vepeside/Etoposide, ZD 9331, Taxotere®/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′ deoxycoformycin), Semustine (methyl-CCNU), Teniposide (VM-26) and Vindesine sulfate.

14. A method for treating cancer in a mammal comprising administering to a mammal having a tumor a compound according to claim 1 in combination with ionizing radiation.

15. The method according to claim 14, wherein the compound according to claim 1 is administered on Day 0 and the radiation is administered as about 3 Gy of radiation on Days 2, 4, and 9.

16. The method of claim 14, wherein the compound is administered from about 1 hour to about 4 days prior to treatment with ionizing radiation.
